(19) United States
(12) Patent Application Publication
(10) Pub. No.: US 2024/0390255 A1
(43) Pub. Date: Nov. 28, 2024

(54) USE OF WATER-SOLUBLE LIGNIN AS AN ANTI-UV FILTER

(71) Applicants: CENTRE NATIONAL DE LA RECHERCHE SCIENTIFIQUE, Paris (FR); UNIVERSITÉ DE BORDEAUX, Bordeaux (FR)

(72) Inventors: Nicolas MANO, TALENCE (FR); Sébastien GOUNEL, BERTHEZ (FR); Philippe CLUZEAU, MONTPON MENESTEROL (FR); Jean-Paul CHAPEL, BORDEAUX (FR)

(21) Appl. No.: 18/694,176

(22) PCT Filed: Sep. 22, 2022
(86) PCT No.: PCT/EP2022/076393

§ 371 Date: Mar. 21, 2024

(30) Foreign Application Priority Data
Sep. 22, 2021 (FR) FR2109967

(51) Int. Cl. A61K 8/72; A61Q 17/04

(52) U.S. Cl. CPC A61K 8/72; A61Q 17/04; A61K 2800/805

(57) ABSTRACT

A water-soluble lignin obtained by enzymatic treatment, the lignin being water-soluble at a pH between 5 and 10, as an anti-UV filter. A cosmetic composition including the lignin.

The present invention relates to the use of a water-soluble lignin obtained by enzymatic treatment, in particular with at least one bilirubin oxidase enzyme, as an anti-UV filter. It also relates to a cosmetic composition comprising such a lignin.

Solar protection is a major concern in the cosmetic industry. In particular, among the different chemical or organic filters used, the stability of these products is an impedance to be complied with to preserve their effectiveness and innocuousness.

For illustration, a very recent study published in Chemical Research in Toxicology (Downs et al, Benzophenone Accumulates over Time from the Degradation of Octocrylene in Commercial Sunscreen Products, 2021, 34, 4, 1046-1054) suggests a harmful effect of the octocrylene during aging thereof, which had not been put forward until now. This study shows that, over time, octocrylene transforms into benzophenone, a compound the authors explain that it is genotoxic, carcinogenic and endocrine disruptor.

Hence, there is a real need to replace this type of anti-UV chemical filters, or at least to reduce the amount thereof in cosmetic products.

Lignin is a natural polymer available in large amounts, in particular by paper or cellulose pulp processing plants. Unlike polysaccharides, this phenolic polymer has been the subject of little industrial value outside the lignosulfonate and of energy valorization applications. Yet, the potential applications of lignin are numerous. Nowadays, commercial lignins are essentially soluble in organic solvents or highly concentrated basic (pH>10) alkaline aqueous solutions. Some lignins are chemically modified and functionalized to give lignosulfonates which are soluble in aqueous solvents with a neutral, acidic or slightly basic pH, but they always contain many impurities.

The patent U.S. Pat. No. 10,729,624 describes the use of mixed microcapsules based on lignins soluble in organic solvents and chemical filters like UV filters. The lignin present in a solvent is dissolved in an aqueous solution with a pH equal to 12, then adjusted to a pH of 7-10. The fact that this lignin is soluble only in a (liposoluble) organic medium considerably limits the amount that could be incorporated into sun creams, and makes their formulation more difficult. Indeed, the large majority of sunscreen products are direct oil-in-water (O/W) emulsions in which the dispersed phase (fatty phase) is a minority.

However, there is no lignin that can be used as such, soluble in a neutral pH aqueous medium, which is effective to protect the surfaces (for example keratin materials such as the skin but also surface coating supports) from UV, in particular the UVA and/or the UVB.

The present invention addresses this problem.

Surprisingly, as shown in the examples, the Inventors show that the water-soluble lignin according to the invention, soluble in water at a pH comprised between 5 and 10, and solubilized in the continuous aqueous phase, clearly improves the anti-UV performances compared to a liposoluble commercial lignin: this water-soluble lignin has a greater protection in the UVB (SPF), but also a greater absorbance in the UVA. In addition, the water-soluble nature allows using this lignin in large amounts, without affecting the stability of the formula. The anti-UV active agents are then better distributed over the keratin materials, after spreading thereof. Finally, this lignin is bio-sourced, and has a certain interest compared to conventional oil-derived chemical anti-UV filters.

Hence, an object of the present invention is the use of a water-soluble lignin obtained by enzymatic treatment, said lignin being soluble in water at a pH comprised between 5 and 10, as an anti-UV filter. Preferably, the enzymatic treatment is performed with at least one bilirubin oxidase enzyme.

The water-soluble lignin obtained by enzymatic treatment, preferably with at least one bilirubin oxidase enzyme, preferably comprising no sulfonate groups or small amounts thereof, is referred to as “the lignin according to the invention”, or “the water-soluble lignin according to the invention”, in the present application.

Another object of the present invention is a cosmetic composition comprising: an aqueous phase comprising at least one water-soluble lignin obtained by enzymatic treatment, said lignin being soluble in water at a pH comprised between 5 and 10, and an oily phase and/or at least one hydrophilic thickening polymer selected from among cellulose and its derivatives, natural gums, scleroglucans, chitin and chitosan derivatives, carrageenans and gellans.

Preferably, the composition according to the invention is substantially free of microcapsules. By “substantially free”, it should be understood that the composition comprises less than 1% by weight relative to the total weight of the composition, preferably less than 0.5% by weight, preferably less than 0.3% by weight, preferably less than 0.1% by weight of microcapsules. Preferably, the composition according to the invention is completely free of microcapsules.

Another object of the present invention is a non-therapeutic cosmetic method for caring for keratin materials, preferably the skin, comprising the application of a cosmetic composition as described hereinbefore over said keratin materials, preferably the skin.

### Water-Soluble Lignin According to the Invention

The water-soluble lignin according to the invention is obtained by enzymatic treatment. This enzymatic modification is specific. Indeed, the obtained lignin differs from a soluble lignin obtained chemically, by the low presence or absence of synthetic impurities by chemical route and the absence of sulfate or sulfonate groups, in particular. Preferably, the enzymatic treatment is performed with at least one bilirubin oxidase enzyme.

The lignin according to the invention is water-soluble. By “water-soluble”, it should be understood that the lignin according to the invention is soluble in an aqueous solution, preferably in water, at a concentration up to 90% by weight relative to the total weight of the solution. The solubility of the lignin is assessed at room temperature, preferably in an aqueous solution, typically water, by microscopy and spontaneous re-dissolution of the lyophilized lignin in an aqueous solvent. No precipitate is observed.

Furthermore, the water-soluble lignin according to the invention is soluble in water at a pH comprised between 5 and 10, preferably between 6 and 8.

Preferably, the water-soluble lignin according to the invention is obtained by a method comprising the following steps:
mixing of the lignin with water preferably comprising a buffer agent,
preferably, ultrasound treatment of the mixture obtained at the end of step a,
enzymatic treatment of the lignin obtained in a or b, with at least one bilirubin oxidase enzyme, in the presence or absence of a redox mediator, to obtain a lignin soluble in an aqueous medium with a pH higher than or equal to 5 and lower than or equal to 10, preferably comprised between 6 and 8, and
separation of the water-soluble lignin from the salts of the mixture and from any insoluble compounds obtained in c, preferably by dialysis or centrifugation.

Typically, a method allowing obtaining the water-soluble lignin according to the invention comprises the solubilization of lignin in a solution comprising water, and preferably also a buffer agent (step a). According to a preferred embodiment, the solubilization of lignin takes place in water as a unique solvent, and preferably with a buffer agent.

According to one embodiment, the lignin is of the kraft (or alkaline lignin) type. We talk about kraft lignin when it is obtained through a kraft process (also known as “kraft pulping” or sulfate process) which is a process of conversion of wood into paper pulp. The kraft process comprises the treatment of wood chips with a hot mixture of water, sodium hydroxide, and sodium sulfide, breaking the wood fibers and separating lignin and hemicellulose from cellulose. This technique comprises mechanical and chemical steps, in particular referred to as impregnation and baking steps.

According to one embodiment, the lignin is of the organosolv type.

For example, a buffer agent is selected from among a borate buffer, a sodium phosphate buffer or any other buffer allowing maintaining a neutral to basic pH.

Preferably, the lignin dispersed in the solution is subjected to an ultrasound treatment (step b), so as to fragment the material into small particles which could better react with the enzymes.

Afterwards, the enzymatic treatment of lignin is performed (step c). This enzymatic treatment of the lignin according to the invention comprises bringing the aqueous solution of dispersed lignin into contact with at least one bilirubin oxidase enzyme (BOD), in the presence or absence of a redox mediator, and obtaining a soluble lignin in a medium with a pH higher than or equal to 6, preferably higher than or equal to 7. Indeed, enzymes of the bilirubin oxidase type, which are stable and active in an acid medium but also in a neutral and basic medium, allow modifying the lignin so as to make it soluble at neutral or basic pH (i.e. at a pH 5 to 10). In addition, these bilirubin oxidases are more active and more stable than laccases at higher temperatures (up to 70° C.).

According to one variant, the bilirubin oxidase (BOD) is fungi-derived. According to one embodiment, the BOD used in the context of the present invention is a BOD described in the international application WO 2012/160517 (BOD E.C. 1.3.3.5. derived from Magnaporthe oryzae).

According to another variant, the BOD is bacteria-derived. According to one embodiment, the BOD used in the context of the present invention is a BOD described in the international application WO 2011/117839 (BOD E.C. 1.3.3.5. derived from Bacillus pumilus).

Typically, bilirubin oxidase is added to the aqueous solution (preferably with a buffer agent) containing lignin, in the presence or absence of a redox mediator, then placed under conditions enabling its enzymatic action on the lignin in order to solubilize it. Typically, bilirubin oxidase is incubated between 20° C. and 80° C., preferably under stirring and oxygen supply. Typically, the enzymatic action takes place for 2 to 48 hours, preferably for 10 to 24 hours.

Among the redox mediators, mention may be made, for example, of 2,2-azino-bis-[3-ethylbenzthiazoline-6-sulfonicacid] ((ABTS), 2,6-dimethoxyphenol (2,6-DMP), Syringaldazine, conjugated or non-conjugated bilirubin, or the compounds of osmiums.

The lignin obtained after enzymatic treatment in aqueous solvent is separated from the salts and from any insoluble compounds, for example by dialysis or centrifugation (step d).

According to a preferred embodiment, the insoluble compounds of the water-soluble lignin are separated, for example by centrifugation. The water-soluble lignin is present in the supernatant when centrifugation is used.

According to one embodiment, the water-soluble lignin is lyophilized.

The water-soluble lignin according to the invention is soluble in water at a pH higher than or equal to 5, preferably higher than or equal to 6, preferably higher than or equal to 7, and lower than 10, preferably lower than 9, preferably lower than 8.

Preferably, the lignin is soluble over a pH range from 6 to 8.

According to a preferred variant, the water-soluble lignin according to the present invention does not comprise sulfonate groups or comprises small amounts of them. By “small amounts of sulfonate groups”, it should be understood a maximum mass content of 1% by weight of sulfonate groups relative to the total weight of lignin. Advantageously, a small amount of sulfonate groups allows conferring unique properties of interest, in particular a very low transmittance, on the water-soluble lignin.

### Use of the Lignin as an Anti-Uv Filter

An object of the present invention is the use of a water-soluble lignin obtained by enzymatic treatment, said lignin being soluble in water at a pH comprised between 5 and 10, as an anti-UV filter. Another object of the present invention is the use of a composition comprising an aqueous phase, said aqueous phase comprising a water-soluble lignin obtained by enzymatic treatment, said lignin being soluble in water at a pH comprised between 5 and 10, as an anti-UV filter. Preferably, the enzymatic treatment is performed with at least one bilirubin oxidase enzyme. Preferably, the water-soluble lignin does not comprise sulfonate groups or small amounts thereof.

Indeed, preferably, the water-soluble lignin according to the invention is solubilized in the aqueous phase of a composition.

This use is applicable in the cosmetic field, in particular for formulating sun creams but also make-up or care compositions; and in other fields. For example, the lignin according to the invention can be used in different materials, in a paint or varnish composition, in a pesticidal composition, in textiles, in concrete or cement compositions, in food films or different coatings or substrates. Indeed, when it is incorporated into a composition or a material or a coating or a substrate, it allows, after application or use, conferring anti-UV properties on said composition or said material or said coating or said substrate.

Preferably, an object of the present invention is the use of a water-soluble lignin obtained by enzymatic treatment, said lignin being soluble in water at a pH comprised between 5 and 10, as an anti-UV filter in cosmetic compositions, for example make-up or care compositions.

Preferably, another object of the present invention is the use of a water-soluble lignin obtained by enzymatic treatment, said lignin being soluble in water at a pH comprised between 5 and 10, as an anti-UV filter in a paint or varnish composition, in a pesticidal composition, in textiles, in concrete or cement compositions, in food films or in different coatings or substrates.

### Cosmetic Composition

The cosmetic composition according to the invention comprises at least one aqueous phase comprising at least one water-soluble lignin obtained by enzymatic treatment, said lignin being soluble in water at a pH comprised between 5 and 10.

Preferably, the enzymatic treatment is performed with at least one bilirubin oxidase enzyme. Preferably, the water-soluble lignin does not comprise or comprises a small amount of sulfonate groups.

Preferably, this composition comprises an aqueous phase in which the water-soluble lignin according to the invention is solubilized, and an oily phase.

In this case, typically, the composition according to the invention is an emulsion. Preferably, the oily phase is dispersed in the aqueous phase, and a direct emulsion (oil-in-water or O/W) is then obtained. The aqueous phase may also be dispersed in the oily phase, and an inverse emulsion (water-in-oil or W/O) is then obtained.

The cosmetic composition according to the invention may also comprise a thickening polymer, to obtain a gelled composition, for example a gel.

### Aqueous Phase

The aqueous phase according to the invention comprises at least water and the water-soluble lignin according to the invention. The water-soluble lignin according to the invention is solubilized in water.

Preferably, the composition according to the invention comprises at least 50% by weight of water relative to the total weight of the composition, preferably at least 60% by weight, preferably at least 70% by weight.

Preferably, the composition according to the invention comprises from 50% to 99% by weight of water relative to the total weight of the composition, preferably from 65% to 95% by weight, preferably from 70% to 90% by weight.

The aqueous phase may also comprise at least one additive selected from among preservatives and humectants. Typically, the preservative may be selected from among parabens and benzyl alcohol. Typically, the humectant may be selected from among polyols, preferably glycerin or sorbitol.

Preferably, the composition according to the invention comprises from 55% to 95% by weight of aqueous phase relative to the total weight of the composition, preferably from 65% to 90% by weight, preferably from 75% to 90% by weight.

Preferably, if the composition according to the invention is in the form of an inverse emulsion, it comprises from 1% to 50% by weight of water relative to the total weight of the composition, preferably from 5% to 45% by weight.

### Water-Soluble Lignin According to the Invention

The aqueous phase comprises at least one water-soluble lignin according to the invention.

The water-soluble lignin according to the invention is as defined before.

Preferably, the composition according to the invention comprises at least 1% by weight of water-soluble lignin according to the invention relative to the total weight of the composition, preferably at least 1.5% by weight, preferably at least 2% by weight.

Preferably, the composition according to the invention comprises from 1% to 30% by weight of water-soluble lignin according to the invention relative to the total weight of the composition, preferably from 1.5% to 25% by weight, preferably from 2% to 20% by weight.

### Hydrophilic Thickening Polymer

The aqueous phase according to the invention may comprise at least one hydrophilic thickening polymer selected from among cellulose and its derivatives, natural gums, scleroglucans, chitin and chitosan derivatives, carrageenans and gellans.

By “hydrophilic thickening polymer”, it should be understood a thickening polymer that is soluble or dispersible in water.

Preferably, said hydrophilic thickening polymer is selected from among:
cellulose and its derivatives, such as carboxymethylcellulose, hydroxymethylcellulose, hydroethylcellulose, hydroxypropylcellulose or hydroxypropylmethylcellulose;
natural gums, such as xanthan gum, guar gum, gum arabic or locust bean gum;
scleroglucans;
chitin and chitosan derivatives;
carrageenans; and
gellans.

Complementarily, besides the thickener hereinabove, the composition according to the invention may comprise a carbomer-type additional thickener.

Preferably, the composition according to the invention comprises an aqueous phase in which the water-soluble lignin according to the invention is solubilized, and at least one hydrophilic thickening polymer selected from among cellulose and its derivatives, natural gums, scleroglucans, chitin and chitosan derivatives, carrageenans and gellans. Such a composition is an aqueous gel.

Preferably, the hydrophilic thickening polymer is present in an amount ranging from 0.1% to 5% by weight relative to the total weight of the composition, preferably from 0.2% to 3% by weight.

### Oily Phase

The composition according to the invention may comprise an oily phase. The oily phase comprises at least one lipophilic compound (i.e. not soluble in water), preferably selected from among oils, waxes, butters and mixtures thereof.

By “oil”, it should be understood a lipophilic compound that is liquid at room temperature.

By “wax”, it should be understood a lipophilic compound that is solid at room temperature.

By “butter”, it should be understood a lipophilic compound that is semi-liquid or semi-solid at room temperature.

The oils, waxes and butters are well known in the prior art. As non-limiting examples, mention may in particular be made of vegetable oils, lanolin, dicaprylyl carbonate, plant-derived butters like shea butter or cocoa butter, or plant-derived or animal-derived waxes, such as for example beeswax.

Preferably, the composition according to the invention comprises from 5% to 50% by weight of oily phase relative to the total weight of the composition, preferably from 10% to 40% by weight, preferably from 15% to 30% by weight.

Preferably, if the composition according to the invention is in the form of an inverse emulsion, it comprises from 50% to 95% by weight of oily phase relative to the total weight of the composition.

Such a composition in the form of an emulsion may also comprise at least one surfactant, at least one lipophilic UV filter and/or at least one coloring material.

### Surfactant

The composition according to the invention may comprise at least one surfactant. Preferably, the surfactant is selected from among anionic surfactants, non-ionic surfactants, amphoteric surfactants and mixtures thereof.

Preferably, the composition according to the invention comprises at least one non-ionic surfactant.

### Lipophilic UV Filter

The composition according to the invention may comprise at least one lipophilic UV filter. Said filter is solubilized in the oily phase.

Preferably, said UV filter is selected from among mineral UV filters, organic UV filters and mixtures thereof.

As a mineral UV filter, mention may be made of cerium oxide, titanium dioxide (TiO₂), zinc oxide or mixtures thereof, preferably titanium dioxide (TiO₂), zinc oxide or mixtures thereof. The mineral UV filter may be functionalized by a surface treatment, for example with a surfactant or with a metal oxide (in particular silica or alumina), in order to confer different properties thereon.

As an organic UV filter, mention may be made of any authorized organic UV filter. Preferably, the organic UV filters that could be used according to the present invention are selected from among benzylidene camphor derivatives, benzophenone derivatives, para-aminobenzoic acid and its derivatives, triazine derivatives, dibenzoylmethane derivatives, salicylic derivatives, cinnamic derivatives, β,β-diphenylacrylate derivatives, phenyl benzimidazole derivatives, phenyl benzotriazole derivatives and benzalmalonate derivatives.

Preferably, the organic UV filter is selected from among camphor benzalkonium methosulfate, ethylhexyl methoxycinnamate, homosalate, para-aminobenzoic acid, isoamyl p-methoxycinnamate, PEG-25 PABA, ethylhexyl dimethyl PABA (or padimate O), octocrylene, anisotriazine, polysilicone-15, drometrizole trisiloxane, oxybenzone (or benzophenone-3), dicamphor terephthalylidene sulfonic acid (or ecamsule), benzylidene dicamphor sulfonic acid, polyacrylamidomethyl benzylidene dicamphor, octyl triazone (or ethylhexyl triazone), butyl methoxydibenzoylmethane (BMDBM or avobenzone), 4-methylbenzylidene camphor, diethylhexyl butamido triazone, diethylamino hydroxybenzoyl hexyl benzoate, phenyl salicylate, ethylhexyl salicylate, disodium phenyl dibenzimidazole tetrasulfonate, methylene bis-benzotriazolyl tetramethylbutylphenol, phenylbenzimidazole sulfonic acid (or ensulizole), benzophenone-4 or 5 and bis-ethylhexyloxyphenol methoxyphenyl triazine.

Preferably, the composition according to the invention comprises at least one lipophilic UV filter selected from among organic UV filters. Preferably, the composition according to the invention comprises at least one lipophilic UV filter selected from among mineral UV filters, preferably from among TiO₂ and cerium oxide, advantageously TiO₂.

Without the intent to be bound by any theory, as shown in the examples, the water-soluble lignin according to the invention has a synergistic effect with a mineral anti-UV filter of the TiO₂ or cerium oxide type, preferably with a mineral anti-UV filter of TiO₂ type.

Preferably, irrespective of the nature of the composition, the additional UV filter(s) is (are) present in an amount ranging from 1% to 20% by weight relative to the total weight of the composition, preferably from 2% to 15% by weight, preferably from 5% to 12% by weight.

### Coloring Material

The composition according to the invention may comprise at least one coloring material.

Preferably, the coloring material is selected from among pigments, nacres and liposoluble dyes.

Preferably, the pigments are selected from among zirconium or cerium oxides, iron or chromium oxides, in particular red iron oxide, yellow iron oxide, brown iron oxide, black iron oxide, manganese violet, ultramarine blue, curcumin, beta-carotene, chlorophyll, chromium hydrate and ferric blue, and mixtures thereof.

Preferably, the nacres are selected from among borosilicates or mica, natural or synthetic, and possibly coated with titanium oxide (TiO₂), iron oxide (Fe₂O₃) (red, yellow or black), chromium oxide (Cr₂O₃) or a mixture of these oxides.

Preferably, the liposoluble dyes are selected from among quinolines, anthraquinones, triarylmethanes, xanthenes, cyanines, phthalocyanines, nitroso, azo and indigoid dyes.

The invention is now illustrated by the following examples.

In the examples, all percentages are given by weight relative to the total weight of the composition, unless indicated otherwise; the temperature is the room temperature (20-25° C.) and expressed in degrees Celsius unless indicated otherwise, the pressure is atmospheric pressure (101,325 Pa), unless indicated otherwise.

### EXAMPLES

### Example 1—Preparation of the Water-Soluble Lignin According to the Invention

The method is described in the application WO2019/076893:

2 g of kraft lignin, purchased from Sigma-Aldrich under the reference (370959), are taken up in 100 mL of 50 mM borate pH9 buffer (step a).

5.2 mg of a Bacillus pumilus BOD are then added to this solution, then the latter is placed at 60° C. under stirring with a magnetized bar at 100 rpm and compressed air bubbling at 0.1 L·min-1, for 16 hours (step c). Afterwards, the solution is dialyzed in a 100 mL of 10 kDa dialysis rod, in three baths of 4 L of water mQ, the first for 2 hours, the second for 4 hours and the third overnight (step d). The solution is centrifuged for 5 minutes at 1,500 rpm.

The supernatant containing the water-soluble lignin according to the invention is lyophilized overnight and stored at room temperature.

### Example 2—Assessment of the Anti-UV Properties of the Water-Soluble Lignin According to the Invention

A reference sun cream (P2) used for the calibration of the in vivo tests for measuring sun protection factors (FPS or Sun Protection Factors-SPF) is herein used as a formula base for testing the effectiveness of lignin (cf. Table 1).

### Composition of the Reference Sun Cream P2

TABLE 1

| | Ingredients | Amounts (%) |
|---|---|---|
| Aqueous phase | Water | Q.S. 100 |
| | Sorbitol | 5 |
| | Triethanolamine | 1 |
| | Methylparaben | 0.3 |
| | Propylparaben | 0.1 |
| | Benzyl alcohol | 0.5 |
| Fatty phase | Lanolin | 4.5 |
| | Cacao butter | 2 |
| | Glyceryl stearate SE | 3 |
| | Stearic acid | 2 |
| | para-amino benzoic acid | 7 |
| | Oxybenzone | 3 |

2% or 4% by weight of the water-soluble lignin according to the invention, relative to the total weight of the composition, are incorporated into the continuous aqueous phase of P2.

Separately, 2% or 4% by weight of a lipophilic commercial lignin (Kraft lignin, Sigma Aldrich—ref 370959) are dissolved in the dispersed fatty phase of P2 in order to compare the two methods and the two lignin types.

SPF measurements have been carried out in vitro on these four creams with the commercial lignin (Table 2) and the water-soluble lignin of the invention (Table 3).

The SPF measurement is carried out in vitro using molded PMMA substrates with a roughness Ra˜4 μm (HelioScreen Labs, HELIOPLATE HD6). The different formulations are then spread over these polymer supports while complying with the protocol of the ISO 24443 standard (Determination of sunscreen UVA photoprotection in vitro). 1.3 mg/cm² of product are then deposited in the form of uniform drops and distributed symmetrically over the substrate. The spreading then continues with small circular movements with the index for less than 30 seconds before reproducing the horizontal back-and-forth movements. Afterwards, the samples are placed in the dark and left to dry for 2 hours. Afterwards, their transmittance is measured using a spectrophotometer provided with an integration sphere. Finally, the SPF is calculated using the following equation:

$$S P {f}_{vitro} = \frac{{\int }_{290 n m}^{400 n m} E ( λ ) \cdot I ( λ ) \cdot d λ}{{\int }_{290 n m}^{400 n m} E ( λ ) \cdot I ( λ ) \cdot T ( λ ) \cdot d λ} \qquad [ Math 1 ]$$

with T(λ) the transmittance at a wavelength λ such that:

$$A ( λ ) = - log ( T ( λ ) ) \qquad [ Math 2 ]$$

with A(λ) the absorbance of the sample, I(λ) the irradiance of the UV source used in vivo and E(λ) the erythema action spectrum.
Assessment of the SPF for the Sun Cream P2 Formulated with 2% and 4% by Weight of the Commercial Lipophilic Lignin, Respectively

TABLE 2

| | | P2 with 2% | P2 with 4% |
|---|---|---|---|
| | | by weight | by weight |
| | | commercial | commercial |
| | P2 | lignin | lignin |
| Average final | 7.2 | 7.1 | 7.6 |
| mass (mg) | | | |
| Average SPF | 20.9 +/− 2.9 | 19.2 +/− 2.2 | 24.2 +/− 2.3 |

Assessment of the SPF for the Sun Cream P2 Formulated with 2% and 4% by Weight of the Water-Soluble Lignin of the Invention, Respectively

TABLE 3

| | | P2 with 2% by | P2 with 4% by |
|---|---|---|---|
| | | weight water- | weight water- |
| | P2 | soluble lignin | soluble lignin |
| Average final | 7.2 | 5.9 | 8.6 |
| mass (mg) | | | |
| Average SPF | 20.9 +/− 4.7 | 24.3 +/− 2.3 | 42.2 +/− 4.9 |

Table 3 shows that the addition of 4% by weight of the water-soluble lignin according to the invention also allows doubling the value of the SPF (20.9 to 42.2).

As shown by these results, the SPF increases more with the use of water-soluble lignin according to the invention in the continuous aqueous phase than with an addition of commercial lipophilic lignin in the dispersed fatty phase, which highlights the interest of the water-soluble lignin.

The comparison of the transmittance spectra shows more generally the interest of using water-soluble lignin, the absorbance of which is much higher than the commercial lignin in the UVA.

Additional formulations of P2, comprising 3% by weight of TiO₂ instead of the two organic filters (para-amino benzoic acid and oxybenzone) of P2 (cf. Table 1), and 2%, 4%, 6% or 8% by weight of water-soluble lignin in the aqueous phase, have been carried out. They comprise the titanium dioxide (TiO₂) mineral filter.

These formulations show the interest of associating the water-soluble lignin with other filters, in order to improve the anti-UV properties of solar products. The obtained results suggest the existence of a synergy between the mineral filters and the water-soluble lignin. Indeed, a reference cream P2 containing, as only filters, 3% by weight of TiO₂ and 6% by weight of water-soluble lignin, allows obtaining an in vitro SPF of more than 34.

The water-soluble lignin according to the invention has several incontestable advantages in particular compared to the liposoluble commercial lignin:

at equal amounts in the formulae, it always provides a significantly higher SPF than the commercial lignin;
the absorbance spectra of the creams formulated with the water-soluble lignin always feature a higher absorbance over the entire UVB and UVA spectrum than those of the creams formulated with the commercial lignin;
the absorbance spectra of the creams formulated with the water-soluble lignin also have a different form, with a much higher absorbance in the UVA. Hence, the water-soluble lignin of the invention has a better protection in the UVA;
the use of the water-soluble lignin facilitates the formulation of the solar compositions, since it enables the incorporation of a greater amount of lignin. In particular, it is possible to exceed 10% by weight, which seems to be impossible with the commercial liposoluble lignin incorporated in the dispersed fatty phase (because this creates to stability and texture problems); and
the fact of incorporating the water-soluble lignin into the continuous aqueous phase allows for a better distribution of the filters, once the cream has been spread over the skin and the evaporated volatile fraction.

### Example 3—Comparison of the Transmittance of the Water-Soluble Lignin According to the Invention with a Chemically-Treated Water-Soluble Lignin

The composition P2 according to Table 1 of Example 2, without the two organic filters, has been mixed with 4% by weight of water-soluble lignin according to the invention relative to the total weight of the composition. Said water-soluble lignin according to the invention has been obtained according to Example 1. Thus, a composition according to the invention is obtained.

The composition P2 according to Table 1 of Example 2, without the two organic filters, has also been mixed with 4% by weight relative to the total weight of the water-soluble lignin composition obtained by chemical treatment. Said water-soluble lignin has been obtained by a sulfite process. It comprises sulfonate groups. Thus, a comparative composition is obtained.

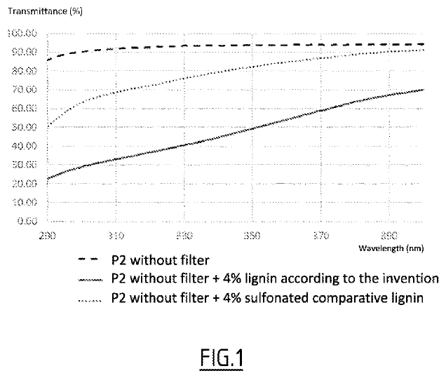

FIG. 1 illustrates the transmittance spectra obtained with the composition P2 as such without the two organic filters (comparative “P2 without filter”), the comparative composition with chemically-treated lignin (“P2 without filter+4% sulfonated comparative lignin”) and the composition according to the invention with enzymatically-treated lignin (“P2 without filter+4% lignin according to the invention”).

The results show that only the composition according to the invention has the lowest transmittance T and therefore a satisfactory absorbance A (A=−log 10 (T)) both in the UVA and in the UVB.

Thus, unlike a chemical treatment, the enzymatic treatment provides unique interesting properties to the water-soluble lignin.

### Example 4—Study of the Combination of Cerium Oxide and of Water-Soluble Lignin According to the Invention

UV absorbance measurements have been carried out on a dispersion of CeO₂ nanoparticles, a solution of enzymatic lignin and a mixture of the two in order to assess the possible synergy of the mixture. For this purpose, 20 ml of a dispersion of CeO₂ nanoparticles at 0.05 g/l and pH=1.5 has been mixed with 20 ml of a solution of enzymatic lignin at the same mass concentration (0.05 g/l) giving a mixture of 0.05 g/l of active agents in total. Afterwards, the pH of the mixture has been adjusted to pH=8 (NaOH) where the new dispersion was stable and transparent. 20 ml of a dispersion of CeO₂ nanoparticles; 2 to 0.05 g/l and pH=1.5 has been adjusted at pH=8 (NaOH). 20 ml of an enzymatic lignin solution at 0.05 g/l has been adjusted at pH=8 (HCl).

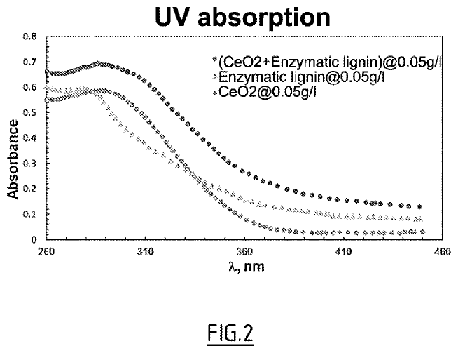

FIG. 2 is a graph showing the evolution of the absorbance of these three solutions as a function of the wavelength. This graph shows that the mixture of the two active agents (CeO₂-lignin complexes) has a higher absorbance than the two active agents considered separately for the same total concentration of active agents of 0.05 g/l, thereby illustrating a synergy effect between the enzymatic lignin and the cerium oxide. This allows reducing the mass concentration of one of the two constituents.

What is claimed is:

1. An anti-UV filter, said filter comprising a water-soluble lignin derived from an enzymatic treatment, said lignin being soluble in water at a pH between 5 and 10, wherein the water-soluble lignin comprises no sulfonate groups or comprises a maximum mass content of 1% by weight of sulfonate groups relative to the total weight of water-soluble lignin.

2. A cosmetic composition comprising:
an aqueous phase comprising at least one water-soluble lignin derived from an enzymatic treatment, said lignin being soluble in water at a pH comprised between 5 and 10, wherein the water-soluble lignin comprises no sulfonate groups or comprises a maximum mass content of 1% by weight of sulfonate groups relative to the total weight of water-soluble lignin; and
an oily phase and/or at least one hydrophilic thickening polymer selected from the group consisting of cellulose and its derivatives, natural gums, scleroglucans, chitin and chitosan derivatives, carrageenans and gellans.

3. The the cosmetic composition according to claim 2, wherein the water-soluble lignin is derived from enzymatic treatment with at least one bilirubin oxidase enzyme.

4. The cosmetic composition according to claim 2, wherein the water-soluble lignin is obtained by a method comprising the following steps:
a—mixing of the lignin with water preferably comprising a buffer agent,
b—preferably, ultrasound treatment of the mixture obtained at the end of step a,
c—treating the lignin obtained in a or b, with at least one bilirubin oxidase enzyme, in the presence or absence of a redox mediator, to obtain a lignin soluble in an aqueous medium with a pH higher than or equal to 5 and lower than or equal to 10, preferably comprised between 6 and 8,
d—separating the water-soluble lignin from the salts of the mixture and any insoluble compounds obtained in c.

5. The cosmetic composition according to claim 3, wherein the bilirubin oxidase is fungi-derived; or the bilirubin oxidase is bacteria-derived.

6. The cosmetic composition according to claim 2, which comprises an aqueous phase in which the water-soluble lignin is solubilized, and an oily phase.

7. The cosmetic composition according to claim 6, wherein the oily phase is dispersed in the aqueous phase, or the aqueous phase is dispersed in the oily phase.

8. The cosmetic composition according to claim 6, which comprises at least one surfactant, at least one lipophilic UV filter and/or at least one coloring material.

9. The cosmetic composition according to claim 6 which comprises at least one lipophilic UV filter selected from among mineral UV filters, organic UV filters and mixtures thereof, preferably from among cerium oxide, titanium dioxide, zinc oxide, benzylidene camphor derivatives, benzophenone derivatives, para-aminobenzoic acid and derivatives thereof, triazine derivatives, dibenzoylmethane derivatives, salicylic derivatives, cinnamic derivatives, derivatives of β,β-diphenylacrylate, phenyl benzimidazole derivatives, phenyl benzotriazole derivatives and benzalmalonate derivatives, more preferably from titanium dioxide, zinc oxide, camphor benzalkonium methosulfate, ethylhexyl methoxycinnamate, homosalate, para-aminobenzoic acid, isoamyl p-methoxycinnamate, PEG-25 PABA, ethylhexyl dimethyl PABA, octocrylene, anisotriazine, polysilicone-15, drometrizole trisiloxane, oxybenzone, dicamphor terephthalylidene sulfonic acid, benzylidene dicamphor sulfonic acid, polyacrylamidomethyl benzylidene dicamphor, octyl triazone, butyl methoxydibenzoylmethane, 4-methylbenzylidene camphor, diethylhexyl butamido triazone, diethylamino hydroxybenzoyl hexyl benzoate, phenyl salicylate, ethylhexyl salicylate, disodium phenyl dibenzimidazole tetrasulfonate, methylene bis-benzotriazolyl, tetramethylbutylphenol, phenylbenzimidazole sulfonic acid, benzophenone-4 or 5 and bis-ethylhexyloxyphenol methoxyphenyl triazine.

10. The cosmetic composition according to claim 2, which comprises an aqueous phase in which the water-soluble lignin is solubilized, and at least one hydrophilic thickening polymer selected from among cellulose and its derivatives, natural gums, scleroglucans, chitin and chitosan derivatives, carrageenans and gellans.

11. The cosmetic composition according to claim 4, wherein separating of the water-soluble lignin from the salts of the mixture and any insoluble compounds obtained in c is performed by dialysis or centrifugation.

12. The cosmetic composition according to claim 5, wherein the fungi-derived bilirubin oxidase is a bilirubin oxidase derived from Magnaporthe oryzae; or the bacteria-derived bilirubine oxidase is a bilirubine oxidase derived from Bacillus pumilus.

13. The cosmetic composition according to claim 5, wherein the bilirubin oxidase is fungi-derived, preferably is a bilirubin oxidase derived from Magnaporthe oryzae; or the bilirubin oxidase is bacteria-derived, preferably is a bilirubin oxidase derived from Bacillus pumilus.

14. The cosmetic composition according to claim 13, wherein the bilirubin oxidase is a bilirubin oxidase derived from Magnaporthe oryzae; or is a bilirubine oxidase derived from Bacillus pumilus.

15. The UV filter according to claim 1, wherein the water-soluble lignin is derived from enzymatic treatment with at least one bilirubin oxidase enzyme.

16. The UV filter according to claim 15, wherein the bilirubin oxidase is fungi-derived, or the bilirubin oxidase is bacteria-derived.

17. The cosmetic composition according to claim 16, wherein the bilirubin oxidase is a bilirubin oxidase derived from Magnaporthe oryzae; or is a bilirubine oxidase derived from Bacillus pumilus.
